(19) United States
(12) Patent Application Publication
(10) Pub. No.: US 2014/0303450 A1
(43) Pub. Date: Oct. 9, 2014

(54) SYSTEM AND METHOD FOR STIMULUS OPTIMIZATION THROUGH CLOSED LOOP ITERATIVE BIOLOGICAL SENSOR FEEDBACK

(71) Applicant: Dylan Caponi, Manhattan Beach, CA (US)

(72) Inventor: Dylan Caponi, Manhattan Beach, CA (US)

(21) Appl. No.: 13/855,780

(22) Filed: Apr. 3, 2013

(51) Int. Cl. A61B 5/16; A61B 5/00

(52) U.S. Cl. CPC A61B 5/165; A61B 5/486
USPC 600/301

(57) ABSTRACT

A system and method for optimizing a stimulus through biological sensor feedback that correlates to the subject's emotional state. Variations on the stimulus to be optimized are presented to the subject and the subject's emotional reaction is evaluated by means of biological sensors. The stimuli that elicit the strongest emotional reaction are then used to generate new stimuli, which are then again presented to the subject until an optimized stimulus is achieved.

### CROSS-REFERENCE TO RELATED APPLICATIONS

The present application claims the benefit of U.S. Provisional Patent Application No. 61/619,910, filed Apr. 3, 2012, which is hereby incorporated by reference in its entirety.

### BACKGROUND OF THE INVENTION

1. Field of the Invention

The present invention relates to media optimization methods, and in particular to a media optimization method based on closed-loop iterative biological sensor feedback evaluating the subject's emotional response.

2. Description of the Prior Art

Marketing and design professionals often use focus groups or test subjects to evaluate and optimize a typical viewer's emotional response to a particular product, logo, advertising jingle, or other stimuli, in an effort to create a stimulus with the maximum emotional impact. Currently, such design processes go through discrete stages—a prototype is developed, then a focus group evaluates the product, the design team analyzes the response of the focus group, creates another prototype based on that response, the focus group evaluates the new prototype, and so on. Alternately, a professional is hired and their aesthetic choices are accepted, vetoed, or modified following the intuition of management. Often times, the product is never evaluated by a proper focus group and the only metric provided are final sales figures.

One problem with this method is that focus group evaluations are by necessity highly subjective because they rely on conscious feedback and are not always detailed enough to be informative on exactly what needs to be changed to improve the product. In areas such as perfume design or music, it requires special training to apply the appropriate vocabulary, and it is often difficult to articulate the root cause of a positive or negative response to a test stimulus. Additionally, the relationship between variables can be complex and interrelated in such a way that one cannot evaluate the impact of altering one property at a time. As a result, design teams often are unable to determine what caused a negative response and what needs to be changed.

One way to solve this problem is by using an optimization algorihm (e.g. an evolutionary or particle search strategy algorithm) to automatically generate new variations on a design, and using the user's feedback to select the variations that impose the greatest desired response from the user, and use these to generate new variations. These methods are often used for cochlear implant fitting; for example, U.S. Pat. No. 6,879,608 to Wakefield et al. discloses such a system, in which a genetic algorithm operates to generate successive generations of multiple groups of values for a parameter subset, and patient feedback determines which half of the group of values are selected and then used to determine the values for the next generation. However, most of those systems and methods are subjective rather than objective, relying on conscious patient feedback via deliberate physical data input. U.S. App. No. 2012/0290045 to Nicolai et al. discloses a similar system, in which one embodiment uses objective rather than subjective measurements; however, the objective measurement is relatively simple and only measures the action potential of the auditory nerve or various latency responses. Usually, such objective measurements are only used for very young or non-cooperative patients, since most patients have no reason to misrepresent the function of their cochlear implant fittings, are easily able to perceive which parameters sound better, and only need to answer simple questions about the loudness of the sound.

The reason such methods have not been used to determine aesthetic appreciation is because a complex stimulus such as a logo or a commercial jingle has many more parameters, and people are often unable to consciously determine how the stimulus affects them. Also, some people may consciously or subconsciously misrepresent their preferences to please the experimenter, to preserve their social image, or for other reasons. As a result, automatic measurements of user perception have been inapplicable in the design and marketing world, and the design process continues to rely on conscious user self-reports. Furthermore, not every user is capable of making conscious reports; very young children, people with disabilities, or animals, are often incapable of expressing their preferences, though they may have them.

An automated method of measuring the user's emotional reaction to complex stimuli and optimizing the creation of said complex stimuli is therefore needed.

### SUMMARY OF THE INVENTION

An object of the present invention is to at least partially optimize an interpreted emotional response of an individual or group by presenting them with an iteratively varied stimulus. The stimulus can be visual (such as a company logo), auditory (such as an advertising jingle), olfactory (such as a perfume), verbal (such as an advertising slogan), or any other type of stimulus that can be perceived by the individual or group.

Another object of the present invention is to use an optimization algorithm (e.g. a genetic algorithm) to iteratively generate new sets of stimuli based on the subject's interpreted emotional responses in order to create a stimulus or stimuli that at least partially achieves the desired emotional response.

A further object of the present invention is to use an optimization algorithm such as a genetic algorithm to iteratively generate new sets of stimuli based on the subject's interpreted emotional responses in order to create an emotional state in the subject.

The present invention provides a system and method for automatically optimizing a stimulus based on the emotional responses of a subject or subjects. In one aspect, the method of the present invention comprises presenting a subject with several initial stimuli one by one, using biological sensors to measure the subject's response to each stimulus, determining the subject's emotional state based on the output of the biological sensors, ranking the stimuli based on the subject's emotional response, selecting one or more highest-ranking stimuli, and using the highest-ranking stimuli to generate new stimuli to present to the subject under test. The steps of presenting the stimuli to the subject, measuring the subject's response, determining the subject's emotional state, ranking the stimuli, and selecting one or more highest-ranking stimuli, are then repeated until the desired level of optimization is achieved. In the preferred embodiment, this is done in real time, and the subject can perceive the stimulus optimizing itself as it happens.

The stimuli can consist of any stimulus or set of stimuli that can be perceived by humans or animals. Any stimulus perceptible by the animal senses can be optimized by the method of the present invention. The stimulus may be visual, auditory, olfactory, tactile, or gustatory, or any combination of the foregoing. For example, the stimuli can be visual art, music, perfume, product design as a rendered 3D model, typography design, industrial design, taste design, slogan design, or any other stimuli that require aesthetic judgment and where it is often difficult to express exact reasons for reacting to a given variation on a stimulus. The stimuli may also be perceptible by the extended senses, such as balance, proprioception, nociception, kinesioception, thermoception, and so on. Stimuli perceptible by multiple senses may also be used, such as videos containing both visual and auditory information.

The biological sensor or sensors used in the method of the present invention can be an EEG, EKG, pneumograph, capnometer, electrodermograph, any other sensor that can measure a biological property of the human or animal body.

The emotional state used in the analysis can be any state that can be reliably correlated to a biological sensor or sensors. For example, excitement, engagement, frustration, meditation, anxiety, happiness, sadness, anger, fear, or any other emotion that can be reliably correlated to biological sensor data can be measured and used in the present invention. The emotional state used in the analysis may also not be correlated to any real emotion, but may simply be a mathematical model or particular combination of the measured responses. The algorithm can utilize a mathematical model (neural networks, state machines, simple mathematical model, etc.) which uses the measured physiological responses of the user to compute a metric which can be defined as an emotion or a pseudo emotion. While it is desirable for this metric to be correlated with an actual human or animal emotion, it is not necessary for the present invention.

The system of the present invention includes one or more biological sensors attached to the subject under test, a display device to display the stimuli to the subject, a computing device that analyzes the output of the biological sensor or sensors and uses the output to calculate the subject's emotional state, and a computing device that selects the stimuli based on the output of the first computing device and implements an optimization algorithm to generate new stimuli based on the selected stimuli. The latter computing device may be the same as the former computing device. The computing solution may be purely hardware or software or a combination thereof.

### DETAILED DESCRIPTION OF THE SEVERAL VIEWS OF THE DRAWINGS

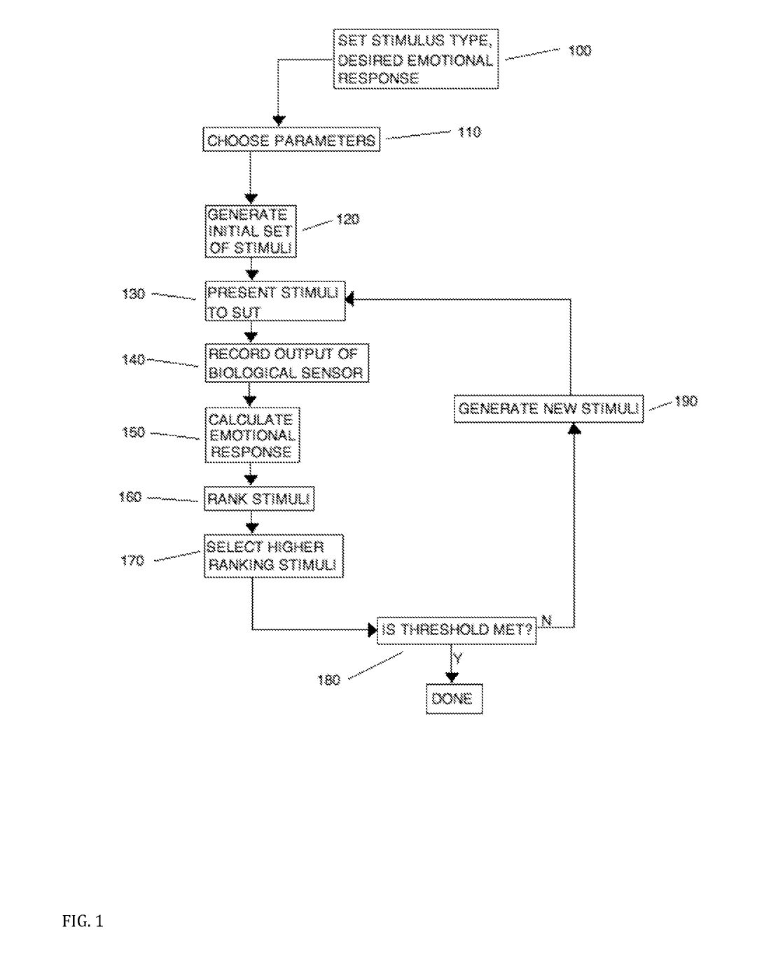

FIG. 1 shows a flowchart of an embodiment of the method of the present invention.

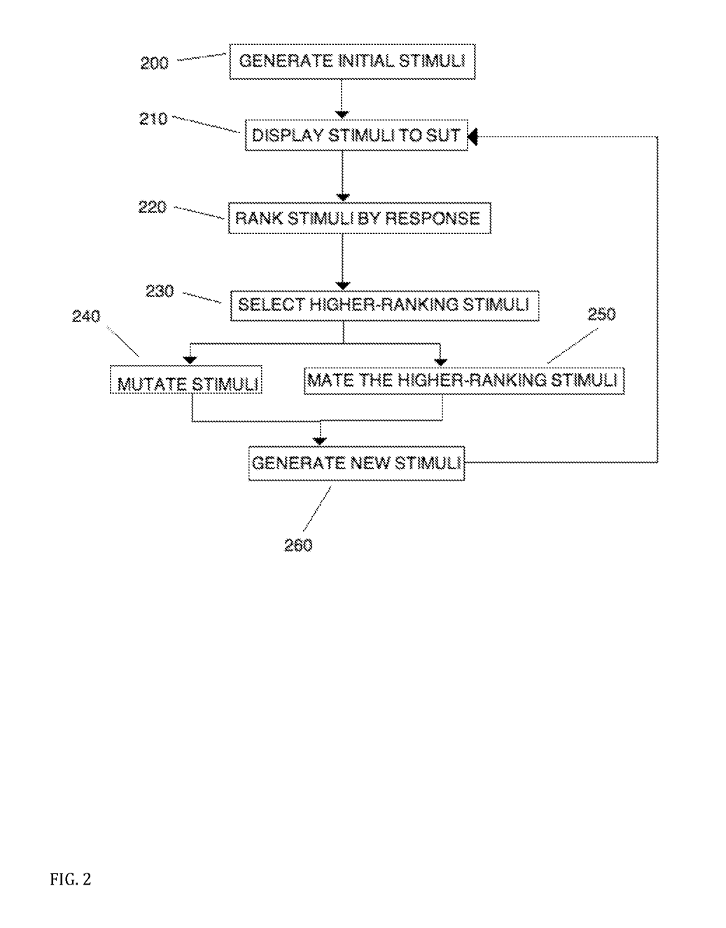

FIG. 2 shows a flowchart of a genetic algorithm used in one embodiment of the method of the present invention.

### DETAILED DESCRIPTION OF THE PREFERRED EMBODIMENT

FIG. 1 shows a flowchart of an embodiment of the method of the present invention. First, a stimulus to be optimized is chosen and a desired emotional or physiological response is set 100. The stimulus can be a product design, a company logo, an advertising jingle, a slogan, a scent, or any other stimulus that can be perceived by a human and that is intended to evoke an emotional response. The emotional response can be excitement, frustration, anger, happiness, engagement, or any other emotion that can be reliably correlated with biological sensor data.

The parameters of a stimulus are chosen by the experimenter 110, and their boundary values or set values are defined. For example, for a logo design, font type and size, color, and text placement may be the variables—then, the boundary values can be the smallest and largest size of the font, the boundary values of the palette of colors to be chosen, and the extreme left, right, top, and bottom positions for placement. Set values, for example, could be a non-continuous set from 1 to 20 in intervals of one and then a jump from 25 to 30 and intervals of 0.1. A set of random initial stimuli is generated based on those parameters 120.

The subject under test (SUT) is then outfitted with at least one biological sensor such as an EEG or EKG. The initial stimuli are then presented to the SUT one by one 130. As the SUT perceives each stimulus, the output of the biological sensor or sensors is recorded by the system and correlated with the appropriate stimulus 140. After exposure to each stimulus, a controllable amount of time passes, and then the system interprets data from the biological sensor or sensors and calculates a rating of how well the stimulus elicited the desired emotional or physiological response (fitness) 150. The stimuli are then ranked by their fitness 160, and one or more highest-ranking stimuli are selected 170.

If the threshold level of fitness has not yet been achieved 180, the optimization algorithm then operates on the highest-ranking stimuli and generates new stimuli from the highest-ranking stimuli 190. The new stimuli are then presented to the SUT 130. The steps of selecting the highest-ranking stimuli and generating new stimuli from the highest-ranking stimuli are then repeated until a threshold level-of-response is met. The system can also be built to stop automatically when the threshold is met. This threshold can be set ahead of time, or determined in real time by the experimenter or the SUT.

In an alternate embodiment of the present invention (not shown), the goal is not to produce an optimized stimulus but rather to produce a desired emotional state in the user—for example, to induce a meditative state. In that case, the steps of selecting the highest-ranking stimuli and generating new stimuli from the highest-ranking stimuli are repeated until the desired emotional state is maintained for the desired amount of time.

In an embodiment of the invention, the emotional responses of several SUTs can be evaluated simultaneously to tailor the mass reaction of the group to the current stimuli.

Any number of highest-ranking stimuli can be selected. The number of highest-ranking stimuli can also be varied as the optimization algorithm progresses.

In one embodiment an optimization algorithm may be a genetic algorithm, as shown in FIG. 2. This is the preferred embodiment of the invention. The optimization algorithm choice can vary depending on what is appropriate for the application as known to those skilled in the art. The genetic algorithm is initialized with a set of random, but parameterized individual stimuli 200. Each individual stimulus is composed of one or more genes, a gene being a representation of one variable used to optimize the stimulus. In the initial set of stimuli, the genes are set randomly. After the initial set of stimuli is displayed to the SUT one by one 210, their fitness level is determined and a specified number of the highest-fitness stimuli proceed to a “mating” phase of the genetic algorithm 250. In that phase, stimuli swap random sections of genes in a process called crossover, or have their genes altered stochastically in a process called mutation 240. New stimuli generated by either one, or both, of these processes, are thus created and make up the next generation of stimuli 260. In one embodiment, a small portion of the previous generation that has the highest fitness is also allowed to pass into the next generation unaltered. The next generation of stimuli is then presented to the SUT, their fitness level is determined, and the highest-fitness stimuli then go through the “mating” phase again. This is repeated until the desired fitness level is reached. As a result, a SUT can watch a logo or a product design modify itself to maximize their emotional reaction in real time.

The “parents” of each individual (i.e. the two stimuli whose genes are swapped to create new stimuli) can be selected randomly, or the probability of each stimulus being selected to be a parent can depend on its fitness level. In other embodiments three or more parents can be chosen to create a single child.

The genetic algorithm may be tailored in several different ways. For example, the number and average span of crossovers, the mutation probability, the selection type, the highest-fitness group size, and the initial population size are all parameters that can be varied depending on the problem at hand. The algorithm may also adjust these parameters dynamically as the optimization process advances.

The applications of the present invention are numerous, and though many are below-listed, many are omitted due to their similarity in terms of product and goal to those already listed. Any product that attempts to elicit an emotional or physiological response by appealing to any of the five traditional senses (or extended senses) to optimize the experience or absence of a currently known (or developed in the future) interpreted emotion or defined physiological state by the use of any biological sensor, can benefit from the use of this invention. Some sample applications include:

a. jingle design,

b. video editing, segment duration, and sequencing

c. organizing advertisement video sequences, selecting video sequences,

d. logo design (font type, style, size, color etc.), company name design

e. word design for visual and auditory aesthetics

f. designing smells

g. designing tastes

h. casting actor combinations

i. avatar aesthetic design

j. cartoon character aesthetic design

k. web page design

l. speech design

m. aesthetic appearances of any product (clothing, electronics, car shapes, accessories),

n. color template design

o. graphical user interface (GUI) design and physical interface design (minimizing frustration)

p. store floor plan layout,

q. physical sensations

r. environmental design

s. virtual/real indoor/outdoor lighting colors

t. magazine covers

u. to optimize a specific physiological state or biological response in a focus group or individual;

v. 3D models for subsequent 3D printing, manufacture, or use in graphics or animations;

w. sexual sensations and stimuli.

We also note that information gleaned from this method can provide valuable statistical data regarding the emotional state of people with regard to stimuli presented. This can allow marketing groups to generate a general understanding (if one exists) of how an individual, or groups of similar individuals, will respond to marketing media. Furthermore, this can be used to understand how biases brought on by cognitive interactions can both positively and negatively influence media design.

Data obtained from this invention can also be used to determine the emotional stability of applicants for high stress quick thinking jobs such as fighter pilots. Potential candidates can be exposed, using this invention as a tool, to a frustrating iterating stimulus such as simulated weather conditions or avionics GUI layout in a flight simulator. The invention can test the candidate's emotional limits and coping mechanisms by attempting to maximize their frustration or fear. An interviewer can make an evaluation or the candidate's flight time can be the objective performance measure.

Another application of the present invention could be videogame difficulty parameters. For example, the AI skill level, number of opponents, level obstacles, and so on, can all be set by the emotional data generated by the biological sensors.

The biological sensors used for the present method can be any sensors that measure a biological phenomenon that can be correlated to an emotion. Some sample sensors that can be used are EEG, EKG, pneumograph (respiration rate), capnometer (CO2 output), or electrodermograph (skin conductance). Other sensors may also be used.

The emotional responses that are evaluated by the present method are any emotions that can be interpreted by biological sensors. For example, the Emotiv EPOC consumer EEG device can measure and rate a SUT's excitement, engagement, frustration, and meditation. Other emotions may also be evaluated by other sensors or by other evaluation systems. For example, an electrodermograph measures skin conductance, which correlates to surprise, arousal, worry, or cognitive activity. A capnometer measures CO2 output, which correlates to stress or anxiety. The vagal tone (the relationship between breathing and heart rate) correlates to happiness, sadness, anger, and fear. Many other emotions have been interpreted by a range of biological sensors and documented in psychological studies.

It is likely that this invention will require SUT specific calibrations. One approach to providing a user baseline would be to expose the SUT to standardized set of relevant stimuli before the stimuli optimization begins. The emotional reactions to these stimuli can serve as a baseline and be compared to other SUT's reactions to help calibrate the system.

Some potential shortcomings of the present invention are the large number of stimuli required to optimize a complex stimulus such as a logo or a jingle, and subject exhaustion to the stimuli. The first problem can be alleviated by limiting the number of variables that can be controlled by the algorithm, thus reducing its search space. The second problem, subject exhaustion, arises when a subject loses interest in the stimuli, or becomes fatigued, after being shown hundreds of pictures or other stimuli. Two ways to counter this problem are limiting the length of stimuli exposure sessions and rating stimuli based on the difference between the current rating and a moving average of the recent history of fitness values or baseline. Those of ordinary skill in the art will recognize that there are a plethora of known weighting methods to utilize, and the disclosed methods are not limited by such variations.

Another shortcoming of the present invention is that if the stimuli are not effective enough to engage the subject, the effect of the stimuli will be less than the noise of the subject's daydreaming or neutral disposition. The stimuli optimized by the present invention must be effective enough to engage the SUT and the SUT has to be attentive to the stimuli.

1. A method, comprising:
generating a plurality of initial stimuli, said stimuli comprising at least one parameter;
presenting a subject with the stimuli;
measuring the subject's response to each stimulus by using at least one biological sensor;
evaluating the subject's emotional state data generated by the biological sensor;
selecting at least one stimulus based on the subject's emotional state;
generating at least one subsequent stimulus;
repeating the presenting, measuring, evaluating, selecting, and generating steps until a desired outcome is reached.

2. The method of claim 1, where the desired outcome is an optimized stimulus.

3. The method of claim 1, where the desired outcome is a modification of the subject's emotional state.

4. The method of claim 1, where the stimuli are selected from the group consisting of:
visual stimuli;
auditory stimuli;
olfactory stimuli;
tactile stimuli;
gustatory stimuli;
thermoceptive stimuli;
proprioceptive stimuli;
nociceptive stimuli;
equilibrioceptive stimuli;
kinesthesioceptive stimuli;
electromagnetic stimuli;
chemical stimuli;
and combinations of the foregoing.

5. The method of claim 1, where the subject's emotional state is selected from the group consisting of:
excitement;
frustration;
depression;
engagement;
meditation;
happiness;
anxiety;
anger;
sadness;
sexual arousal;
and combinations of the foregoing.

6. The method of claim 1, where the biological sensor is selected from the group consisting of:
an electrocardiograph;
an electroencephalograph;
a pneumograph;
a capnometer;
an electrodermograph;
a penile tumescence sensor;
an oxygen sensor or optode via pulse oximetry;
a camera or other device for measuring papillary response;
a camera or facial electromyograph for measuring and interpreting facial expressions;
a microphone to measure changes in spoken vocal properties such as pitch, inflection, and speed;
and combinations of the foregoing.

7. The method of claim 1, where the generating step is performed by a genetic algorithm.

8. The method of claim 1, where the selecting step comprises:
ranking the stimuli according to the magnitude of the subject's response;
selecting at least one highest-ranking stimulus.

9. The method of claim 1, where the repeating step is repeated until a threshold level-of-response is met.

10. The method of claim 6, where the optimization algorithm comprises the following steps:
assigning a variable to each parameter of the stimulus;
swapping random sections of variables between at least one stimulus and at least one other stimulus;
creating new stimuli based on the swapped variables.

11. The method of claim 9, further comprising:
stochastically altering at least one variable in at least one stimulus.

12. The method of claim 9, where the at least one stimulus and at least one other stimulus are selected randomly.

13. The method of claim 9, where the at least one stimulus and at least one other stimulus are selected based on ranking.

14. The method of claim 1, where the selecting step comprises:
ranking the stimulus based on a moving average of the recent history of rankings;
selecting at least one highest-ranking stimulus.

15. A system, comprising:
at least one biological sensor that can sense a biological property of a human or animal subject;
a presentation device for presenting at least one type of stimulus to the subject;
computing means for determining an emotional state of the subject based on the output of the at least one biological sensor;
computing means for ranking the stimuli presented to the subject based on the emotional state;
computing means for generating new stimuli based on the higher-ranked stimuli.

16. The system of claim 14, where the at least one biological sensor is selected from the group consisting of:
an electrocardiograph;
an electroencephalograph;
a pneumograph;
a capnometer;
an electrodermograph;
a penile tumescence sensor;
an oxygen sensor or optode via pulse oximetry;
a camera or other device for measuring papillary response;
a camera or facial electromyograph for measuring and interpreting facial expressions;
a microphone to measure changes in spoken vocal properties such as pitch, inflection, and speed;
and combinations of the foregoing.

17. The system of claim 14, where the stimuli are selected from the group consisting of:
visual stimuli;
auditory stimuli;
olfactory stimuli;
tactile stimuli;
gustatory stimuli;
thermoceptive stimuli;
proprioceptive stimuli;
nociceptive stimuli;
equilibrioceptive stimuli;
kinesthesioceptive stimuli;
and combinations of the foregoing.

18. The system of claim 15, where the computing means is a computer comprising a processor and memory.
